(19) United States
(12) Patent Application Publication
(10) Pub. No.: US 2008/0293804 A1
(43) Pub. Date: Nov. 27, 2008

(54) Pharmaceutical Nasal Spray Formulation of Acetaminophen (paracetamol)

(76) Inventor: Muwaffak Jeryis Haddadin, Monmouth JCT, NJ (US)

Correspondence Address: Muwaffak J Haddadin, 106 Rotal Oaks CT, Monmouth JCT, NJ, 08852, US

(21) Appl. No.: 12/104,292

(22) Filed: Apr. 16, 2008

(30) Foreign Application Priority Data
Jan. 8, 2007 (JO) 618

(51) Int. Cl. A61K 31/405; A61K 31/167; A61K 31/192

(52) U.S. Cl. 514/420; 514/629; 514/570

(57) ABSTRACT

The invention in question is a novel pharmaceutical formulation that comprises a one percent weight by volume Paracetamol in a phosphate buffer adjusted to pH 7.4 by addition of few drops of 1.0 N NaOH. The concentrations of the ingredients were chosen such that the resulting solution is isotonic with body fluids. Said concentrations were theoretically calculated based on their sodium chloride equivalents. The formulation is meant to be used as a nasal spray.

### DETAILED DESCRIPTION OF THE INVENTION

### Introduction

Many formulations and medications were introduced to the market to treat migraine and headaches. The route of such drugs is either oral or parenteral except for a few nasal preparations of other active pharmaceutical ingredients which are prescription items. When taken orally, their onset of action needs some time. When taken parenterally, a healthcare provider must administer and/or supervise administration. This means inconvenience and more cost to the patient.

### ART OF THE CLAIM

What is new in the formulation in question, are the following:

FIG. 1. Isotonic with body fluids.

FIG. 2. Buffered at physiological pH of 7.4.

FIG. 3. Application of its active ingredient(s) as a nasal spray.

These three properties are not known for commercial paracetamol formulations or similar analgesics. Furthermore, it is expected that the formulation will be licensed as an OTC.

### METHOD OF PREPARATION

One gram (gm) of pharmaceutical grade paracetamol is dissolved in about 70 milliliter (mL) of pharmaceutical grade phosphate buffer and made up to 100 mL with same buffer. The final pH was adjusted to 7.4 by addition of a few drops of 1.0 N sodium hydroxide. Alternatively components of the buffer can be adjusted to arrive at this pH. The buffer is prepared by dissolving 0.6 gm of sodium acid phosphate and 0.8 gm of sodium triphosphate in 70 mL of distilled or purified water then quantum sufficit (Q.S.) to 100 mL. The weights of the buffer components were arrived at by using the Henderson-Hassaelbach equation. The main ionic species of this buffer will be sodium acid phosphate whose sodium chloride equivalent (0.4) and that of paracetamol (0.3) are used to calculate the required weights to arrive at isotonic solution.

1. Composition of the preparation:

| | |
|---|---|
| a. Paracetamol: | 1.0 gram |
| b. Sodium Acid Phosphate: | 0.6 gram |
| c. Trisodium Phosphate: | 0.8 gram |

2. The pH of preparation was adjusted with 1.0 N sodium hydroxide to the physiological pH (7.4). Other options of pH can take a range 5.5 to 6.7, which is the range of pH of nasal mucus.

3. The isotonicity of human body fluids was achieved by using the above composition (based on upon theoretical calculations).

4. Application of above preparation in the form of nasal spray.

5. Slight Modifications, to preserve the pH and isotonicity, of above composition can be made to accommodate for other analgesics like (but not limited to ibuprofen, ketoprufen, indomethacin, narcotic, and non-narcotic analgesics alone or in combination).
